(19) United States
(12) Patent Application Publication
(10) Pub. No.: US 2023/0017228 A1
(43) Pub. Date: Jan. 19, 2023

(54) METHOD OF LYOPHILIZATION OF A CRYOGENIZED CELLULAR COMPOSITION CONTAINING DISSOLVED GAS

(71) Applicants: GENIALIS, HENRICHEMONT (FR); LARENA, OREE D'ANJOU (FR)

(72) Inventors: Guillaume GILLET, SALBRIS (FR); Sarah KUYLLE, CARBONNE (FR); Francois PAUL, MONTGAUCH (FR); Manon CAYET, SEYSSES (FR)

(21) Appl. No.: 17/780,336

(22) PCT Filed: Dec. 2, 2020
(86) PCT No.: PCT/FR2020/052241

§ 371 Date: May 26, 2022

(30) Foreign Application Priority Data
Dec. 2, 2019 (FR) FR1913627

(51) Int. Cl. C12N 1/04; C12N 1/20

(52) U.S. Cl. CPC C12N 1/04; C12N 1/20

(57) ABSTRACT

The invention relates to the field of lyophilizates of biological materials. More particularly, the invention relates to a new method for preparing a lyophilizate of cells comprising a step of cryogenics “under pressure”. In a preferred embodiment, this method is applied to lactic acid bacteria.

The invention relates to the field of lyophilizates of biological materials. More particularly, the invention relates to a new method for preparing a lyophilizate of cells comprising a cryogenics step “under pressure”. In a preferred embodiment, this method is applied to lactic acid bacteria.

### STATE OF THE ART

Storage of cells in frozen form has long been known. This method requires little equipment. The principle is simple: freezing causes a temperature drop, which slows and then stops all of the cell's biochemical reactions. The latter are capable of being reactivated after thawing. However, whatever method is used, it is found that there is a significant loss of viability on thawing, caused in particular by the formation of intracellular crystals, and the longer the duration of the freezing phase, the more this is the case.

Alternatively, the cells may be lyophilized. Lyophilization is a method of drying a previously frozen product, by sublimation. This method therefore gives a dry product that can be stored at 4° C. or at ambient temperature. However, this method is long, expensive and very energy-consuming.

Conventionally, a method of lyophilization of cells comprises the steps of preparing a sample comprising the cells, centrifugation of the sample and taking up the cellular pellet in a cryoprotective medium allowing dehydration, freezing of the sample and then lyophilization. The freezing step is generally carried out at temperatures comprised between −20° C. (slow freezing) and −80° C. (quick freezing); quick freezing is less harmful to the cells but more difficult to implement on an industrial scale.

There have been attempts to freeze cells by cryogenics, alone or with a view to lyophilization, but the results proved rather unconvincing owing to considerable deterioration of the cells. This deterioration probably results from the fact that the methods of the prior art do not allow control of the quantities of water and oxygen present in the product during the freezing and lyophilization steps. Water and oxygen are two factors that affect the quality of the cells and their long-term viability. Thus, none of the cryogenics solutions proposed to date have made it possible to improve the properties of the lyophilized products significantly. In particular, most of the solutions proposed are based on a lowering of the pressure, applied during the freezing step. However, application WO2018/138461 describes a method of cooling biological material by cryogenics at high pressure, namely at a pressure comprised between 10 and 1000 bar. This method allows an improvement in the survival rate of a strain of Lactobacillus bulgaricus by a factor of almost 400.

Among the many drawbacks associated with the methods available to date, it is regrettable not to be able to obtain preparations of lactic acid bacteria of probiotic interest having long-term stability at ambient temperature.

More generally, there is a need for a method for long-term storage of cells of all types, in particular of live cells.

### DISCLOSURE OF THE INVENTION

The inventors have shown that the quality and the long-term viability of lyophilized cells, in particular of probiotic bacterial strains, may be improved significantly when the freezing step is carried out by cryogenics under pressure, allowing dissolution of gases within the matrix to be frozen before lyophilization.

Thus, the invention relates to a method of lyophilization of a cellular composition, characterized in that the freezing step is coupled with considerable dissolution of gases in the product, taking place just before said freezing. Dissolution is effected either by carrying out the freezing in a chamber under pressure, or by subjecting the product to a very high density of gas molecules but without an increased pressure necessarily being observed. These two embodiments are equivalent in terms of result, in the sense that the matrix passes through a dense zone of gas molecules (step of dissolution of gases in the matrix) before being frozen by cryogenics.

### ADVANTAGES OF THE INVENTION

The inventors have demonstrated the advantage of cryogenics under pressure as a method for freezing biological materials, especially bacteria such as lactic acid bacteria.

In fact, this method allows better preservation of the cells during the freezing and lyophilization steps and therefore an improvement in production yields of frozen cellular preparations.

Firstly, it is found that cryogenics under pressure is itself advantageous with respect to the other methods of freezing for preservation of the integrity and/or viability of cells.

Moreover, it is very advantageous to combine a step of freezing by cryogenics under pressure with lyophilization, for several reasons:
The production yield is better; a lyophilized composition richer in cells is recovered at the end of the process. Consequently, the cell concentration in the lyophilizate is improved;
The cells are less damaged, and their membrane integrity is maintained; this is advantageous for preserving cell viability, but also in the case of damaged or dead cells, the membrane or wall of which has been damaged. In fact, it was shown several years ago that non-viable bacteria may retain certain probiotic activities (adhesion, stimulation of the immune system etc.).
The viability of the cells is increased at the end of the lyophilization step compared to products obtained by the conventional methods of lyophilization.
Stability of the cell lyophilizates is also observed in the long term, namely for several months, at 4° C. and at ambient temperature.

The advantages associated with this method are observed when the initial cellular composition contains cryoprotectants. Interestingly, they may also be observed in the absence of cryoprotectants with certain cells, in particular certain bacteria; elimination of the use of cryoprotectants limits the addition of additives, which are increasingly being disparaged.

Moreover, this method makes it possible to treat matrices for which the size of the beads formed may reach 7 to 8 mm as well as aggregates; the size parameter affects neither the efficacy of the method, nor the quality of lyophilized products obtained.

At the end of this method, it is proposed to package the lyophilizates under a protective atmosphere, in particular to avoid the alteration caused by oxidation. In order to limit contact with oxygen as much as possible, it is advantageous to carry out scavenging of the lyophilizate with an inert gas before sealing the packaging, in order to further increase the stability of the lyophilizates over time. With this procedure, enhanced stability is expected, which may reach more than a year.

Thus, by incorporation of gas coupled with very quick cooling making it possible to keep the gas dissolved and maintain anaerobiosis throughout the process, the quality of the lyophilized cells is improved considerably.

The method according to the invention makes it possible to control the three critical parameters linked to the quality of a preparation of lyophilized cells: cell concentration, oxidation and the quantity of free water (the presence of water being harmful vis-à-vis the stability of lyophilized cells at ambient temperature and at 4° C.), and thus permits the preparation of lyophilizates of bacteria of probiotic interest the properties of which are preserved in the long term. Storage at 4° C. is more or less suitable, depending on the product. In the case of preparations of bacteria of probiotic interest, storage at ambient temperature makes them easier to use (transport, storage, use). It is therefore advantageous to have preparations of bacteria of probiotic interest that can be stored at ambient temperature.

This method may be applied to many cellular types: bacteria of probiotic interest and for the food industry (used for cheesemaking in particular), yeasts, plant cells, microalgae, microorganisms from the intestinal or faecal microbiota (for transplantation of faecal microbiota in the treatment of infections with Clostridium difficile), reproductive cells (oocytes and spermatozoa) for animal and human reproduction, blood cells and stem cells.

Without being bound to this theory, the inventors think that the benefits of the method according to the invention for preparing frozen and/or lyophilized cells are based on the fact that cryogenics “under pressure” makes it possible to obtain non-porous frozen products containing a large quantity of perfectly dissolved gas. As this gas is not oxygen, oxidation reactions are avoided. Moreover, lyophilization of such products makes it possible to remove most of the water contained in the product. As a result, the conditions implemented in this method are on the whole milder, less aggressive and less destructive for the cells.

This method makes it possible to lyophilize fragile bacterial strains, which cannot withstand conventional lyophilization conditions.

It also makes it possible to prepare bacterial lyophilizates that are stable at temperatures of 20° C., or even 25° C. for long periods of up to 24 months in order to meet the expectations of industrial companies wishing to have lyophilized products that are more resistant to temperature changes so as to reduce stresses during transport, storage and preservation.

From the standpoint of the method of lyophilization as such and industrialization thereof, the cryogenics step “under pressure” also gives consequent improvements. Firstly, the preparation time is much quicker. Cryogenics is an almost instantaneous process, making it possible to produce, continuously and at high rates (several hundred kg per hour with existing equipment), beads of a product that is initially fluid. The time gain is considerable with respect to freezing in a refrigerating chamber, even if the latter operates at very low temperatures (generally −40° C. to −80° C.). The cryogenized beads are extracted at temperatures generally comprised between −80° C. and −120° C., which makes it possible to start lyophilization directly, with products the temperature of which is close to −60° C., without a preliminary cooling step. The lyophilization time itself is reduced considerably (at least by a factor of 2). Finally, this method makes it possible to treat larger quantities of raw materials by reducing the treatment time. This last-mentioned advantage makes it possible to obtain quality products even in the absence of cryoprotectant.

This method opens up new prospects for the storage of cells in the form of lyophilizate, the properties of which are preserved during the lyophilization process and restored after redissolving, both at the level of the active molecules and at the level of the viability of whole cells.

### DETAILED DESCRIPTION OF THE INVENTION

The invention relates firstly to a method of lyophilization of a cellular composition, characterized in that the freezing step is carried out by cryogenics, and which comprises the steps of:
a) providing a cellular composition comprising cells in an aqueous medium;
b) dissolving a gas in said composition by passage through a dense zone of gas molecules, such a density being obtained (i) either owing to the flow of gas generated by the evaporation of a cryogenic fluid, (ii) or by raising the pressure, (iii) or by a combination of both phenomena;
c) cryogenizing said gas-rich composition obtained in step b) at a pressure that makes it possible to keep said gas dissolved for obtaining frozen granules, particles or beads;
d) lyophilization of said frozen granules, particles or beads to obtain a lyophilized cellular preparation.

This method is therefore characterized in that the matrix constituted by a cellular composition is frozen by cryogenics in contact with a gas so as to dissolve gas in said matrix. Dissolution is obtained by passing said matrix through a dense zone of gas molecules.

The zone rich in gas molecules inside the chamber containing the cryogenic gas may be obtained in three ways: i) either owing to the flow of gas generated by the evaporation of a cryogenic fluid, (ii) or by raising the pressure, (iii) or by combining the two phenomena.

When the dense zone of molecules is obtained owing to a flow of gas generated by the evaporation of a cryogenic fluid, the quantity of gas dissolved in the matrix is typically equivalent, when no pressure is applied, to what would be obtained by the application of relative pressures comprised between 0.001 bar and 2 bar. This condition is called “cryozero” in the experimental section.

When the dense zone of molecules is obtained by raising the pressure, this pressure is above atmospheric pressure, and may in particular be above 0.1 bar, 0.5 bar, 1 bar, 2 bar, 5 bar, 10 bar, 15 bar, 20 bar, 25 bar, 30 bar, 50 bar or 100 bar.

Within the meaning of the invention, by the term “under pressure” is meant conditions that allow dissolution of a gas in a matrix and/or keeping the gas dissolved in said matrix during deep-freezing. Pressurization may be obtained either by raising the pressure, or by contacting the matrix with a cryogenic fluid, evaporation of this gas creating a density of gas molecules equivalent to pressurization so that the gas molecules dissolve in the matrix, or by a combination of the first two phenomena. Moreover, “pressurization” corresponds to the application of relative pressures, i.e. atmospheric pressure is regarded as a pressure of 0 bar. All the pressures expressed in the present document are relative pressures. In a preferred embodiment of the invention, the method is not carried out under partial vacuum.

Thus, step b) is carried out at a relative pressure sufficient to allow dissolution of gas in the matrix and an equivalent pressure is maintained in step c) to keep the gas dissolved in the matrix during cryogenization.

In a particular embodiment of the invention, the pressure applied in step c) is greater than or equal to atmospheric pressure.

The gas may be an inert gas. The gas may be nitrogen, nitrous oxide, carbon dioxide, a rare gas such as argon or a mixture of these gases.

In a preferred embodiment, the gas used is nitrogen, and the cryogenic fluid is liquid nitrogen.

Regarding the overall implementation of the method, it is possible to perform the steps of the method one after another and in particular carry out the lyophilization step immediately after the cryogenics step. Moreover, the method may be carried out continuously. It is also possible to store the product in the frozen form at the end of step c) and carry out lyophilization subsequently, after a time of cold storage at negative temperature to keep the products in the solid state (for example at −40° C.). In both cases, the advantages of the method are maintained.

The conditions of the method may be adapted as a function of the product to be dehydrated, in particular the pressure in the cryogenics step, and the lyophilization parameters. A person skilled in the art will know how to carry out these adaptations.

By “cell” is meant, within the meaning of the invention, a prokaryotic or eukaryotic cell. Among the prokaryotic cells, there may be mentioned lactic acid bacteria, bacteria of probiotic interest, bacteria making up the microbiota (intestinal, faecal etc.). Among the eukaryotic cells, there may be mentioned yeasts, reproductive cells, blood cells and stem cells, plant cells and microalgae. The cells treated by the methods described in the present document may be living, or not. They may in particular be tyndallized bacteria, i.e. killed by heat beforehand. The cells may be isolated from one another or may be organized in the form of tissues.

In a preferred embodiment, the cellular composition is constituted by bacteria of probiotic interest for humans and animals. In another preferred embodiment, the cellular composition is constituted by a sample of faecal microbiota.

By “aqueous medium” is meant water, a culture medium, an aqueous plant extract. This aqueous medium may or may not contain cryoprotectants.

Thus, in a particular embodiment, the cellular composition does not contain a cryoprotectant.

The invention relates secondly to a method for preparing a composition of cells frozen by cryogenics under pressure comprising the steps of:
a) providing a cellular composition comprising cells in an aqueous medium in the form of a matrix;
b) dissolving a gas in said matrix by passage through a dense zone of gas molecules, such a density being obtained (i) either owing to the flow of gas generated by the evaporation of a cryogenic fluid, (ii) or by raising the pressure, which may be up to 10 bar, (iii) or by combining a flow of gas as mentioned in (i) with raising the pressure;
c) cryogenizing said gas-rich matrix obtained in step b) at a pressure that makes it possible to keep said gas dissolved in said matrix to obtain frozen granules, particles or beads.

This method makes it possible to obtain frozen cells the survival of which is increased with respect to the methods of freezing described previously.

The pressure applied in step b) is below 10 bar, but may in certain embodiments be below 5 bar, or even below 2 bar.

In a preferred embodiment, the pressure that makes it possible to keep said gas dissolved in said matrix is greater than or equal to atmospheric pressure.

The invention relates thirdly to a lyophilized cellular composition obtained by the method of lyophilization as defined above.

In a particular embodiment, the composition comprises a lyophilizate and dehydrated maltodextrins.

In a particular embodiment, the lyophilized cellular composition comprises lyophilized and stabilized probiotic bacteria of interest obtained by the method as defined above and dehydrated maltodextrins. In a preferred embodiment, such a composition comprises 20-25% lyophilizate and 75-80% dehydrated maltodextrins.

The present invention will be better understood on reading the examples given hereunder, provided by way of illustration and not in any way to be regarded as limiting the scope of the present invention.

### DESCRIPTION OF THE FIGURES

FIG. 1: Viability of the Lactobacillus plantarum ATCC SD-5209 strain after freezing by cryogenics “under pressure”.

FIG. 2: A—Concentrations of CFUs and ICs in the lyophilizates of the Lactobacillus plantarum BL3504 strain. B—Yields in viability and ICs of the lyophilizates of the Lactobacillus plantarum ATCC SD-5209 strain. C—Cytometric profiles of the lyophilizates of the Lactobacillus salivarius BL2201 strain.

FIG. 3: A—Loss of viability under conditions of accelerated ageing (30° C./65% RH) of the lyophilizates of the Bifidobacterium animalis ssp lactis BL3803 strain over 3 months. B—Loss of viability under conditions of accelerated ageing (30° C./65% RH) of the lyophilizates of the Lactobacillus plantarum BL3504 strain over 12 months.

FIG. 4: Comparison of the yields of intact cells of the lyophilizates of a Lactobacillus plantarum ATCC SD-5209 strain with or without cryoprotectant.

### EXAMPLES

### Example 1

### Effect of Cryogenics on the Properties of a Frozen Composition of Lactic Acid Bacteria The samples are prepared from the commercial strain Lactobacillus plantarum ATCC SD-5209.

The bacteria are solubilized in a reconstituted vegetable MRS medium without carbohydrate+sucrose 1%+maltodextrins 5% (cryoprotectants), so that the concentration of intact cells is adjusted to 5.10⁹ cells/mL. The −80° C. control corresponds to freezing of 40 mL at −80° C. for a minimum of 48 h. The −20° C. control corresponds to freezing of 40 mL at −20° C. for a minimum of 48 h. For the strains frozen by cryogenics, different pressures were tested. The samples are obtained using equipment making it possible to implement the method as described in patent EP07858410, at a relative pressure of 8 bar in the cryogenics chamber. At the end of the freezing step, all the samples are stored in a chamber at −80° C. before being analysed. The samples are then thawed (1 h at 37° C.) and the viability is measured.

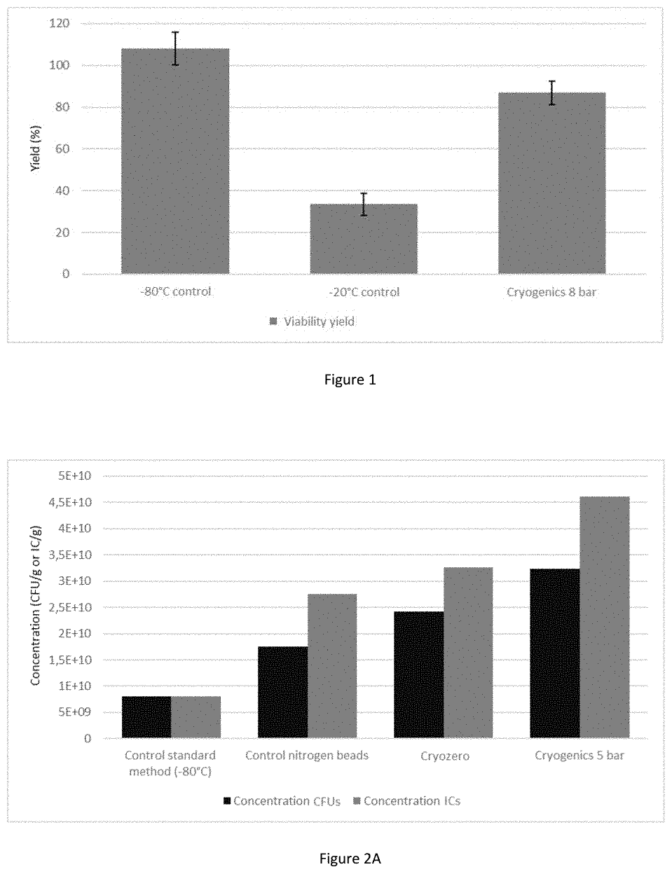

The results obtained are presented in FIG. 1.

It can be seen that the loss of viability of the control frozen at −20° C. is far greater than for the sample frozen by cryogenics at 8 bar. This result is entirely significant and shows a real benefit from cryogenics.

### Example 2

### Effect of Cryogenics on the Properties of a Lyophilized Composition of Lactic Acid Bacteria

A—Investigation of a Freshly Cultured Composition of Lactobacillus plantarum BL3504

A bacterial sample is cultured for 24 h in 500 mL of culture medium of the vegetable MRS type (Ref BK176HA from Biokar Diagnostics). The culture medium is then centrifuged for 10 min at a speed of 3400 g.

The pellet, containing the microorganisms under investigation, is taken up in fresh culture medium so that the concentration of intact cells is adjusted to 5.10⁹ cells/mL.

The preparation is then separated into 4 fractions making it possible to test the 4 different methods of freezing:
The sample “control standard method” is prepared by aliquoting the preparation in 30 mL bottles and placing the latter in a chamber at −80° C. for at least 48 h so as to guarantee that all of the product has frozen and has reached a temperature of −80° C.;
The sample “control nitrogen beads” is prepared by manually producing a drip, using a syringe, above an open vessel, such as a basin or a bowl, containing liquid nitrogen, at atmospheric pressure and ambient temperature;
The sample “cryogenics 5 bar” is obtained using equipment making it possible to implement the method as described in patent EP07858410, at a relative pressure of 5 bar in the cryogenics chamber;
The sample “cryozero” is obtained using equipment making it possible to implement a cryogenics step at a relative pressure of 0 bar, in such a way that the saturation of dissolved nitrogen obtained is about 3 times greater than would be obtained at this same pressure with the “control nitrogen beads”.

At the end of the freezing step, all the samples are stored in a chamber at −80° C. before being analysed.

The samples frozen by the 4 methods are also lyophilized, in a Drywinner CT60 lyophilizer (Heto Holten) for 48 h to 72 h. The samples are ground carefully using a pestle and mortar to reduce them to powder. They are then stored in sterile pots at −20° C. before being analysed. For the analyses, the powder samples are first dispersed in buffered peptone water with addition of Tween 80 at 1% (w/v) and homogenized using a Stomacher. The quantities of CFUs and ICs are obtained by counting in MRS agar and by cytometric analysis (protocol B of standard ISO 19344 IDF 232 v2015).

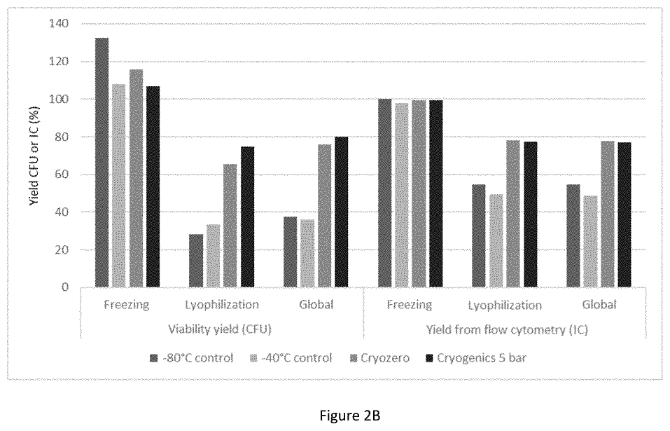

The results in FIG. 2-A were obtained with the Lactobacillus plantarum BL3504 strain.

This chart shows the results obtained according to 2 parameters:
the concentration of live cells in the lyophilizate, in CFUs (viability test consisting of testing the capacity of the bacteria to form colonies on an agar medium),
the membrane integrity of the cells in the lyophilizate, expressed as intact cells (ICs) measured by flow cytometry.

The results show that the use of a faster cooling rate (cryogenics: “control nitrogen beads”) makes it possible to double or triple the concentrations of CFUs and ICs in the lyophilizate with respect to the “control standard method (−80° C)”. The “cryozero” condition makes it possible to increase these concentrations further, since a tripling or quadrupling of the concentrations of CFUs and ICs of the “control standard method (−80° C.)” is achieved. Finally, the “cryogenics 5 bar” condition makes it possible to reach the highest concentrations of CFUs and ICs, 4 and 6 times higher than those in the standard method.

B—Investigation of a Commercial Composition of Lactobacillus plantarum ATCC SD-5209

The samples were prepared from the commercial strain Lactobacillus plantarum ATCC SD-5209.

The bacteria are solubilized at a level of 10% dry matter in buffered peptone water (tryptone 1.0g/L+NaCl 8.5 g/L+K₂HPO₄ 2.5 g/L+KH₂PO₄ 2.5 g/L) with addition of 5% (w/w) maltodextrins (cryoprotectant). The −80° C. control corresponds to freezing of 10 mL at −80° C. for a minimum of 48h. The −40° C. control corresponds to freezing of 10 mL at −40° C. for a minimum of 48 h. For the cryogenically frozen samples, various pressures were tested. The samples are obtained using equipment making it possible to implement the method as described in patent EP07858410, at a relative pressure of 0 and 5 bar in the cryogenics chamber (“cryozero” and “cryogenics 5 bar” samples). The cryogenically frozen samples are stored in a chamber at −80° C. before being analysed. Some of the samples are then thawed (30 min at ambient temperature) and the viability is measured.

The samples frozen according to the 4 methods are in addition lyophilized, in a Drywinner CT60 lyophilizer (Heto Holten) for 72 h. The samples are ground carefully using a pestle and mortar to reduce them to powder. They are then stored in an aluminium sachet at −20° C. before being analysed. For the analyses, the powder samples are first dispersed in buffered peptone water with addition of Tween 80 at 1% (w/v) and homogenized using a Stomacher. The quantities of CFUs and ICs are obtained by counting in MRS agar and by cytometric analysis (protocol B of standard ISO 19344 IDF 232 v2015).

The results obtained are presented in FIG. 2-B.

This chart presents 3 yields, calculated either from the concentration of live cells measured by viability (CFUs), or from the concentration of intact cells measured by flow cytometry (ICs):
“Freezing” yield: concentration in the frozen samples relative to the concentration before freezing,
“Lyophilization” yield: concentration in the lyophilizates relative to the concentration in the frozen samples,
“Global” yield: concentration in the lyophilizates relative to the concentration before freezing.

The results show that the use of cryogenics as a method for freezing samples of lactic acid bacteria makes it possible to increase the yields in viability and in intact cells at the level of the lyophilization step as well as in the overall method. In fact, the yields after the freezing step are not affected by the method used: 100% yield of CFUs and ICs is obtained whatever the sample. The positive effect of freezing by cryogenics clearly occurs during lyophilization with yields from 70 to 80% against 30 to 50% for the −40° C. and −80° C. controls. This effect is also observed for the method as a whole (global yield). However, these results do not make it possible to discriminate the two conditions of cryogenics: the same yields are obtained for the “cryozero” and “cryogenics 5 bar” samples. It may therefore be deduced from this that cryogenics under pressure under the conditions of molecular density described above, is one means for better preserving the intact cells and therefore improving the viability of samples of lactic acid bacteria produced by lyophilization.

C—Investigation of a Composition of Lactobacillus salivarius BL2201

The samples are prepared from the Lactobacillus salivarius BL2201 strain, according to the method described in Example 2-B.

The results obtained are presented in FIG. 2-C.

This chart presents the composition of the samples as intact cells, damaged cells and dead cells, measured by flow cytometry, at each step of the method (sample before freezing, frozen sample and lyophilizate).

The aim of these tests is to demonstrate the advantage obtained by freezing by cryogenics under pressure, even for a strain regarded as fragile, since the initial preparation used contains 45% damaged and dead cells. The results obtained also show that the effect from cryogenics occurs at the level of the lyophilization step. In fact, the cytometric profile of the different frozen samples is the same as that of the preparation before freezing, with about 55% intact cells. However, differences are observed in the cytometric profiles of the lyophilized samples: the lyophilizates of the −80° C. and −40° C. controls contain 4 and 5% ICs respectively, whereas the lyophilizates of the samples cryogenized under cryozero and 5 bar conditions contain 13% ICs. It can therefore be concluded that carrying out the freezing step by cryogenics “under pressure” is one means for preserving the level of intact cells in a lyophilized preparation of a fragile strain that does not easily withstand the standard method of lyophilization.

### Example 3

### Effect of the Method of Lyophilization on the Viability of a Composition of Lyophilized Bacteria (Dry Powder) Over Time

A—Investigation of the Stability of a Freshly Cultured Composition of Bifidobacterium animalis spp lactis BL3803

The samples are prepared by the method described in Example 2-A. The lyophilizates in powder form are stored for 3 months, after dilution in dehydrated maize maltodextrins, in paper/aluminium/PE three-layer sachets and kept at 30° C. and 65% RH before being analysed. For the analyses, the samples in powder form are first dispersed as stated in Example 2-A. The quantities of CFUs are obtained by counting in MRS agar, at t=0 just after production of the samples, then after storage for 1.5 months and 3 months, respectively. The loss of viability is calculated by difference between the quantity of CFUs at time t and that corresponding to t=0.

The experiments were carried out using the Bifidobacterium animalis spp lactis BL3803 strain.

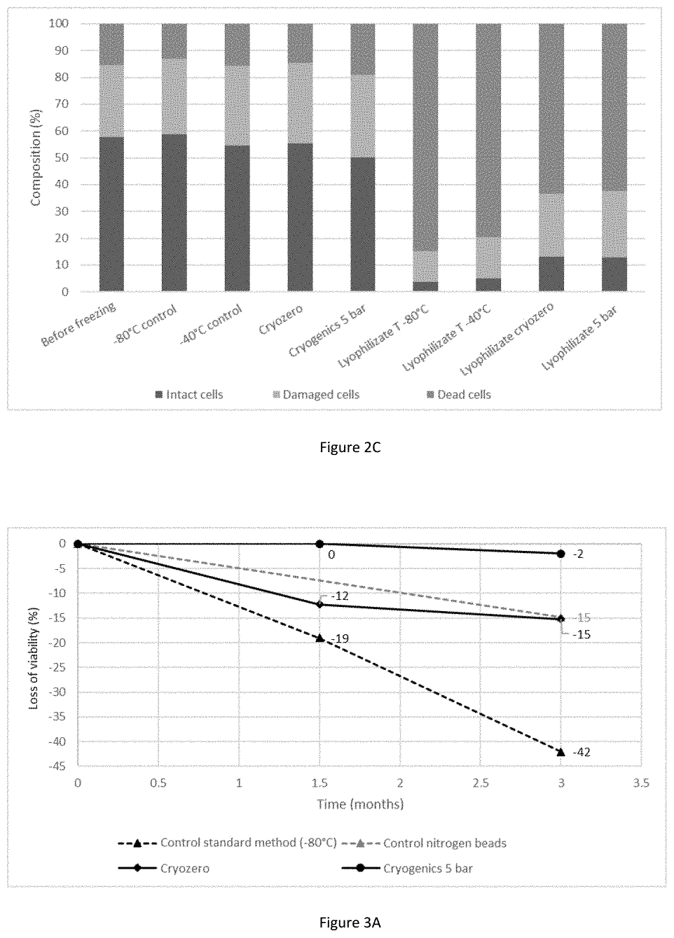

The results obtained are presented in FIG. 3-A.

It can be seen that the loss of viability is considerable under the standard conditions (“−80° C. control”) since this loss is nearly half of the CFUs present initially. Conversely, the loss observed for the sample obtained from cryogenics at 5 bar is very low and close to 0. The results obtained for the “control nitrogen beads” and for the “cryozero” condition are comparable and intermediate, which also shows the positive effect of cryogenics “under pressure” on the method proposed for preservation of strains in lyophilized form.

B—Investigation of the stability of a freshly cultured composition of Lactobacillus plantarum BL3504

The samples are prepared by the method described in Example 2-A. The lyophilizates in powder form are stored for 12 months, after dilution in dehydrated maize maltodextrins, in paper/aluminium/PE three-layer sachets and kept at 30° C. and 65% RH before being analysed. For the analyses, the samples in powder form are first dispersed as stated in Example 2-A. The quantities of CFUs are obtained by counting in MRS agar, at t=0 just after production of the samples, and then after 1.5 months, 3 months, 4.5 months, 6 months, 9 months and 12 months of storage. The loss of viability is calculated by difference between the quantity of CFUs at a time t and that corresponding to t=0.

The experiments were carried out using the Lactobacillus plantarum BL3504 strain.

The results obtained are presented in FIG. 3-B.

It can be seen that the loss of viability is considerable under the standard conditions (“−80° C. control”) since this loss is already above 50% after 1.5 months of storage and the percentage of viable cells is less than 20% starting from 4.5 months. Conversely, the loss observed for the samples obtained from cryogenics is much slower. It is only significant (>20%) starting from the sixth month and about 60% of the cells are still viable after 12 months of storage. For comparison, barely more than 10% of the cells are still viable under the conditions of freezing at −80° C. The results obtained for the “control nitrogen beads” and “cryogenics under pressure” are comparable.

The loss of viability is slow enough under the three conditions for the difference between them not to be significant.

### Example 4

### Effect of the Presence of Cryoprotectant on the Membrane Integrity of a Lyophilized Composition of Lactic Acid Bacteria

The samples are prepared from the commercial strain Lactobacillus plantarum ATCC SD-5209, by the method described in Example 2-B, with or without cryoprotectants (maltodextrins 5% (w/w)).

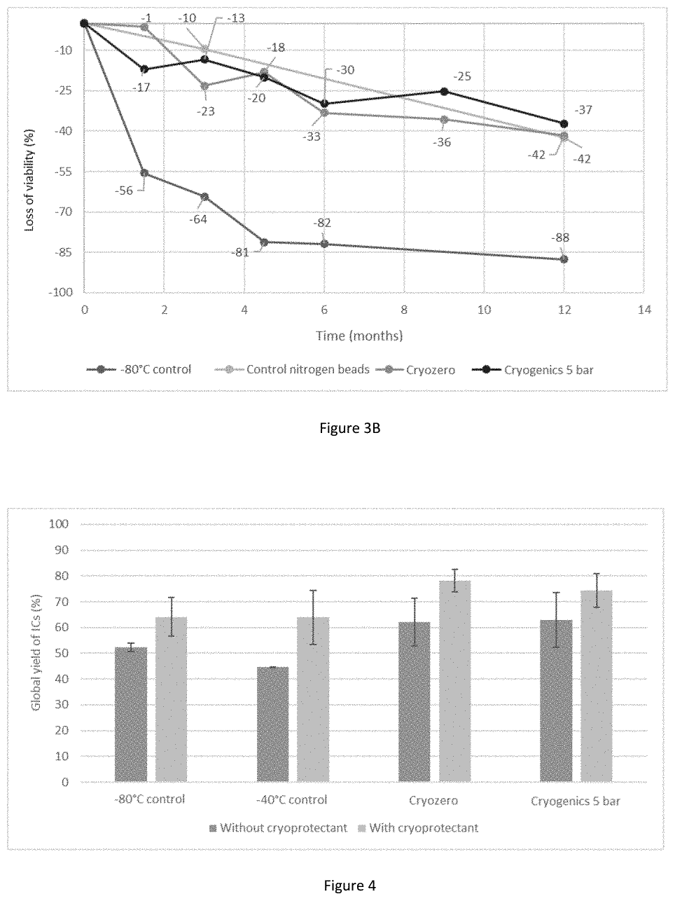

The results obtained are presented in FIG. 4.

This chart presents the yields of the overall method, calculated from the concentration of intact cells (ICs) in the lyophilizates (measured by flow cytometry) relative to the concentration before freezing. The tests were done in triplicate, and error bars corresponding to the standard deviation of each condition are shown.

These results show that the presence of cryoprotectants in the samples allows better preservation of the intact cells during the freezing and lyophilization process. In fact, higher yields of ICs are observed for the samples containing maltodextrins, whatever the freezing conditions. It can therefore be deduced from this that the combined use of cryoprotectants and cryogenics gives an improvement in the quality of the lyophilizates of lactic acid bacteria.

Conclusion: The method of lyophilization of a cellular composition comprising a freezing step carried out by cryogenics “under pressure” gives a both qualitative and quantitative improvement of the production of cell lyophilizates.

1. A method of lyophilization of a cellular composition, characterized in that the freezing step is carried out by cryogenics under pressure, said method comprising the steps of:
a) providing a cellular composition comprising cells in an aqueous medium;
b) dissolving a gas in said composition by passage through a dense zone of gas molecules, such a density being obtained (i) either owing to the flow of gas generated by the evaporation of a cryogenic fluid, (ii) or by raising the pressure, (iii) or by the combination of the two phenomena;
c) cryogenizing said gas-rich composition obtained in step b) at a pressure that allows said gas to be kept dissolved in said composition for obtaining frozen granules, particles or beads;
d) lyophilization of said frozen granules, particles or beads to obtain a lyophilized cellular preparation.

2. A method for preparing a composition of cells frozen by cryogenics under pressure comprising the steps of:
a) providing a cellular composition comprising cells in an aqueous medium in the form of a matrix;
b) dissolving a gas in said matrix by passage through a dense zone of gas molecules, such a density being obtained (i) either owing to the flow of gas generated by the evaporation of a cryogenic fluid, (ii) or by raising the pressure, which may be up to 10 bar, (iii) or by combining a flow of gas as mentioned in (i) with raising the pressure;
c) cryogenizing said gas-rich matrix obtained in step b) at a pressure that allows said gas to be kept dissolved in said matrix for obtaining frozen granules, particles or beads.

3. The method of claim 1, in which said gas is nitrogen.

4. The method of claim 1, in which said cellular composition comprises lactic acid bacteria of probiotic interest, bacteria forming the microbiota, yeasts, plant cells, microalgae, reproductive cells, blood cells, stem cells.

5. The method of claim 4, in which said cellular composition comprises bacteria of probiotic interest for humans and animals.

6. The method of claim 1, in which the cellular composition does not contain cryoprotectant.

7. The method of claim 4, in which said cellular composition is a sample of faecal microbiota.

8. A lyophilized cellular composition obtained by the method of claim 1.

9. The composition of claim 8, further comprising dehydrated maltodextrins.

10. The composition of claim 8, in which the cellular composition comprises bacteria of probiotic interest.

11. The method of claim 2, in which said gas is nitrogen.

12. The method of claim 2, in which said cellular composition comprises lactic acid bacteria of probiotic interest, bacteria forming the microbiota, yeasts, plant cells, microalgae, reproductive cells, blood cells, stem cells.

13. The method of claim 12, in which said cellular composition comprises bacteria of probiotic interest for humans and animals.

14. The method of claim 2, in which the cellular composition does not contain cryoprotectant.

15. The method of claim 12, in which said cellular composition is a sample of faecal microbiota.
